(19) United States
(12) Patent Application Publication
(10) Pub. No.: US 2018/0140245 A1
(43) Pub. Date: May 24, 2018

(54) SYSTEMS AND METHODS FOR IDENTIFICATION AND PREDICTION OF STRUCTURAL SPINE PAIN

(71) Applicant: Keijo Tapio Videman, Chapel Hill, NC (US)

(72) Inventor: Keijo Tapio Videman, Chapel Hill, NC (US)

(21) Appl. No.: 15/356,553

(22) Filed: Nov. 19, 2016

(51) Int. Cl. A61B 5/00; A61B 5/055; A61B 5/103

(52) U.S. Cl. CPC A61B 5/407; A61B 5/055; A61B 2576/02; A61B 5/0022; A61B 5/103

(57) ABSTRACT

Systems, computer-readable media, and methods for assessing morphometric measures of spinal vertebrae and inter-vertebral discs in a quantitative manner using imaging data and human body weight measures, for identification and prediction of structural spine pain, are described. The systems, media, and methods of the present disclosure utilize simple measurements of axial areas of vertebrae using endplate data of routinely acquired digital imaging pixels and body weight. More specifically, they provide a calculation pressure value based on one or more ratios from body weight and measurements of spinal structures and regions, which are readily determinable, e.g., through manual segmenting or automated programming of image analysis software.

### FIELD

The present disclosure relates to systems, computer-readable media, and methods for assessing morphometric measures of spinal vertebrae and inter-vertebral discs in a quantitative manner using magnetic resonance imaging (MRI) data and human body weight measures, for identification and prediction of structural spine pain. The systems, media, and methods of the present disclosure utilize simple measurements of axial areas of vertebrae and data of routinely acquired digital MRI pixels and body weight. More specifically, they provide a calculation pressure value, e.g., of five axials' mean area between external upper and lower epiphyseal rings (axial vertebra area′), based on one or more ratios from body weight and measurements of spinal structures and vertebra regions, which are readily determinable, e.g., through manual segmenting or automated programming of image analysis software.

### DESCRIPTION OF THE RELATED ART

Common spinal disorders associated with intervertebral disc degeneration, such as disc herniation and spinal stenosis, have been a major focal point of research and clinical investigations into structural spine pain problems. Despite the extent of this effort, it currently is estimated that more than 80% of the patients seeking care for idiopathic back pain complaints cannot be given a precise pathoanatomical diagnosis.

Structural spine pain in the lumbar spine has been variously characterized in research efforts and clinical practice as “idiopathic back pain”, “degenerative disc disease (DDD)”, “non-specific back pain”, and “discogenic pain” but precise criteria for characterization have proven elusive. Intervertebral disc area “instability pain” has been a rational explanation for idiopathic back pain. It has been speculated that idiopathic back pain may be a non-fully-adaptive physiological artifact of bipedal evolution. Most back pain studies since the 1950s have been directed to pathoanatomical indicia of disc degeneration as an etiological basis for assessment, but these efforts have simply failed to provide an objective basis for diagnosis and interventional therapy. Early efforts to link structural spine pain to neurological correlates were complicated by the fact that non-ruptured discs have nerves only on the outer disc surface. Disc narrowing, disc bulging and vertebral osteophytes gradually emerged as primary indicators of disc degeneration in assessments of spinal health, and signal loss was added as a characteristic following the adoption of MRI techniques using hydration reflecting sequences, such as T2-weighted images. Today, most spine studies are based on the views that back pain derives from degenerative disc changes such as disc height decrease, disc bulging and disc dehydration

Although disc degeneration is viewed as a causal factor in the majority of back pain presentations, challenges have existed in the ability to separate normal aging degeneration from pathologic degeneration (Weiner, B. K., Spine 2008, 8:925-30). In this respect, many organs and other physiological systems exhibit aging degenerative changes that are not associated with incidence of pain until progression to inflammation, rupture, or a catastrophic episode such as cardiovascular failure. Classical spinal health studies have failed to show association between back pain and disc degeneration with clinical values (Borenstein, D., O'Mara, J. W., et al., J. Bone Joint Surg. Am., 2001, 83-A:1306-11).

The reported investigations in the field have included studies of back pain and vertebra associations in vertebra endplate injuries (Wang, Y., Videman, T., et al., Spine 17, 1976, 1490-6; and Teraguchi, M., Yoshimura, N., et al., Spine J., 2015, 15:622-628), and some studies have associated Modic type endplate changes with back pain (Jarvinen, J., Karppinen, J., et al., BMC Musculoskelet. Disord., 2015, 22:16:98; and Weiner, B. K., et al., Eur. Spine J., 28:2014;). A twin study (Videman, T., Battié, M. C., et al., Spine, 2003, 28:582-8) investigated associations of twelve common suspected risk factors with current, past 12 month, and lifetime back pain reports concluded that: “[a]fter taking the combined effects of genetics and shared environment into account, disc height and tears each explained 7% and 6%, respectively, of the total variance in the LBP [low back pain] in the past year”; “[e]ndplate irregularities, osteophytes, bulging, herniations, spinal stenosis, and the signal intensity of the disc did not enter into the model's explaining any of the pain parameters”; and “[t]hese findings raise new questions about the underlying mechanisms of LBP”.

Pressure in the spine due to body weight and behavioral and external physical activities has been of interest among back disorder researchers; in 1955 the anatomist Goff concluded that “[t]hese mechanical strains have contributed to many types of low backache as ‘fatigue’” and “[v]ertical compression forces may be major factors in producing spondylolisthesis, herniated discs . . . ” (Goff, C. W. Clin. Orthop.1955:5:8-16). Nachemson (Nachemson, A. Acta Orthop Scand, 1959:28:269-89) published the first related article in 1959, and pressure in the spine based on body weight and behavioral and external physical activities has been of interest among back disorder researchers since that time. Wilke and Neef et al. (1999) confirmed earlier study, and provided the following pressures: lying prone 0.1 MPa; relaxed standing 0.5 MPa; standing flexed forward 0.46 MPa; sitting with maximum flexion 0.83 MPa; nonchalant sitting 0.3 MPa; and lifting a 20-kg weight with round flexed back 2.3 MPa (Wilke, H. J., Neef, P., et al. Spine, 1999:15:755-62). In 2007 Nachemson concluded that “[i]t [pressure] does not give any indication as to where the pain actually comes from” (The BackLetter 2007; 22:13-21).

Even though the pressure itself does not have a directional character, it relates to the forces perpendicular to the surrounding surfaces. If body weight increases over time, the endplate areas of the vertebrae increase, but at a rate much more slowly than typical increases in weight. The endplate area increase can be explained by Wolff's law: increasing the area decreases the pressure on the area (Julius Wolff, German anatomist, 1836-1902).

In sum, after more than 60 years of clinical studies, research, observations, and reports, the numerous identified suspected risk factors for structural spine pain have failed to provide any meaningful consensus concerning the etiology, progressionary characteristics, and useful diagnostic and prognostic approaches associated with structural spine pain. The interventional therapies that have been proposed have shown results that are not significantly better than placebo (Cherkin, D., Deyo, R., et al., NEJM, 1998, 339:1021-9). The dominant view that idiopathic back pain derives from the inter-vertebral discs has thus far failed to be substantiated by any rigorous investigations.

It would therefore be a dramatic advance in the art to provide reliable methods and tools for quantitative characterization, rigorous diagnosis, and longitudinally accurate prognosis of structural spine pain.

### SUMMARY

The present disclosure relates to systems, computer-readable media, and methods for assessing morphometric measures of spinal vertebrae and inter-vertebral discs in a quantitative manner using imaging data, e.g., magnetic resonance imaging (MRI) data, and human body weight measures, for identification and prediction of structural spine pain.

Although the ensuing description in the disclosure is primarily directed to imaging and imaging data generated by magnetic resonance imaging (MRI) apparatus, it will be understood that all such references to MRI imaging, apparatus, and imaging data are equally applicable to other imaging systems, equipment, processes, and data, and other imaging apparatus and techniques may be employed for spinal imaging in accordance with the disclosure. Such alternative imaging equipment and techniques include, without limitation, ultrasound, tomography (including micro-computed tomography (micro-CT) and x-ray microtomography, as well as computed tomography (CT) generally), emission, fluorescence, and other radiative, absorptive, emissive, and resonance technologies, as well as any other methodologies that are effective and useful in the systems, computer-readable media, and methods of the present disclosure, to provide three-dimensional measurements and characterization of vertebrae. Currently, routine MRI is produced without any radiation, and is conventionally used in hospitals and other medical facilities.

In one aspect, the disclosure relates to a computer-implemented method of quantitatively determining susceptibility to structural spine pain (idiopathic back pain) in a vertebrate subject, such method comprising: generating or obtaining an image, which may for example be a routine magnetic resonance imaging (MRI) image, of the spine or a selected portion thereof; determining by computer-implemented determination from the image, using the discs' mean axial areas as a proxy of vertebra area, a spinal pressure loading value based on body weight of the vertebrate subject (it being noted that routine MRI practice does not provide vertebra areas); and generating by computer-implemented generation from the spinal pressure loading value, a prognostic characterization of the susceptibility of the vertebrate subject to structural spine pain, optionally as age-adjusted for the vertebrate subject.

In another aspect, the disclosure relates to an MRI imaging and computational system for determining susceptibility of a vertebrate subject to structural spine pain, such system comprising: an imaging apparatus, e.g., an MRI apparatus, configured to generate an image of a mean axial area locus of the vertebrate subject; and a computer or other processor programmably configured to determine from data of the image generated by the imaging apparatus, a proxy vertebra axial area locus, to determine from the proxy vertebra axial area a spinal pressure loading value based on body weight of the vertebrate subject, and to responsively generate from the spinal pressure loading value, a prognostic characterization of the susceptibility of the vertebrate subject to structural spine pain.

In a further aspect, the disclosure relates to a method of quantitatively determining susceptibility to structural spine pain of a human patient, comprising operating a digital computational apparatus in accordance with a programmed set of instructions to (i) determine from imaging data of an image of vertebra axial locus and weight of the human patient, a spinal pressure at the vertebra axial locus, and (ii) generate from the determined spinal pressure at the axial vertebra locus a prognostic characterization of susceptibility of the human patient to structural spine pain, optionally as age-adjusted for the human patient.

A further aspect of the disclosure relates to a computer-implemented method of quantitatively determining susceptibility to structural spine pain in a vertebrate subject, said method comprising: generating or obtaining an image of a spinal external epiphyseal ring of a spinal axial vertebra area locus; determining by computer-implemented determination from data of the image, a proxy endplate area of the spinal axial vertebra area locus; determining by computer-implemented determination from the proxy endplate area, a spinal pressure loading value based on body weight of the vertebrate subject; and generating by computer-implemented generation from the spinal pressure loading value, a prognostic characterization of the susceptibility of the vertebrate subject to structural spine pain.

The disclosure relates in another aspect to a non-transitory computer-readable storage medium storing instructions executable by a computer system to prognostically characterize susceptibility of the vertebrate subject to structural spine pain, the non-transitory computer-readable storage medium storing instructions to conduct a method of the disclosure, e.g., as described above.

In yet another aspect, the disclosure relates to an imaging and computational system for determining susceptibility of a vertebrate subject to structural spine pain, said system comprising: an imaging apparatus configured to generate an image of a spinal axial vertebra area locus of the vertebrate subject; and a computer or other processor programmably configured to determine from data of the image generated by the imaging apparatus, a proxy endplate area of the spinal axial vertebra area locus, to determine from the proxy endplate area a spinal pressure loading value based on body weight of the vertebrate subject, and to responsively generate from the spinal pressure loading value, a prognostic characterization of the susceptibility of the vertebrate subject to structural spine pain.

Other aspects, features and embodiments of the disclosure will be more fully apparent from the ensuing description and appended claims.

### BRIEF DESCRIPTION OF THE DRAWINGS

FIG. 1A and FIG. 1B are MRI images of respective spines exhibiting degeneration. In FIG. 1A, the highest two and lowest discs are severely degenerated. In FIG. 1B, the lowest disc is healthy and the three above are degenerated, which is a common degenerated state.

FIG. 2A is a cryomicrotome figure showing three lumbar spine levels, in which the upper disc is healthy, and the lower disc is severely degenerated. The darker areas in the disc are gel (mainly glycosaminoglycan). FIG. 2B is an axial disc image on which a white line has been manually traced along the disc's outer boundary.

FIG. 3 is a sagittal histologic image of an inter-vertebral disc on which the superimposed dotted line represents the axial length between bony endplate ends, wherein the bony endplate ends represent clear stable points of with pressure area. The darker arrows point to bony endplates and the lighter arrows point to cartilaginous endplates.

FIGS. 4A and 4B are sagittal MRI images, illustrating how vertebra endplate area is measured. In FIG. 4A, the mid-sagittal vertebra image is supplemented by 4 arrows pointing to axial slices that are close to the endpoint areas, however these produce errors due to disc height variation. In FIG. 4B, the mean of the 5 dashed lines (shown as AB, 34, 56, 78, DC) is used as the mid width of that vertebra. The upper linear horizontal black line 1-2 in each instance is used as the mean disc diameter.

FIGS. 5A-5D illustrate the challenges of obtaining accurate area measurements. FIG. 5A shows the vertebra's upper osseous endplate as healthy. FIG. 5B shows the vertebra having a deep concave endplate. FIG. 5C shows the vertebra having a large osteophyte rim around the edge of the endplate (osteophytosis). FIG. 5D shows the vertebra endplate to be flat but exceptionally rough.

FIG. 6 is an MRI image of a spine with an exceptionally large bulging anteriorly (to the left) in the middle lumbar spine, as an example of axial increase of endplates.

FIG. 7 is a top plan view of a spinal vertebra, showing the epiphyseal ring (ER) of the vertebral body, as defined by the inner tracing line (I) and the outer tracing line (O).

FIG. 8 is a schematic representation of a magnetic resonance image processing system according to one embodiment of the present disclosure.

### DETAILED DESCRIPTION

The present disclosure relates to systems, computer-readable media, and methods for assessing morphometric measures of spinal vertebrae and inter-vertebral discs in a quantitative manner using imaging data, e.g., magnetic resonance imaging (MRI) data, and human body weight measures, for identification and prediction of structural spine pain.

The systems, computer-readable media, and methodologies of the present disclosure reflect the discovery that susceptibility to structural spine pain (as provoked by loading) increases with decreasing surface area (surface area typically does not decrease in normal aging) of the vertebrate, because with any given level of loading, the pressure in the vertebra is inversely proportional to mean axial area.

Historically, it took half a century of physiological investigation to determine that vertebrae have an important role in structural spine pain, but the specific locus constituting the origin of structural spine pain was still undetermined. It also was known that structural spine pain occurred in various instances in childhood and was a precursor of structural spine pain in later years.

Concerning physical loading of the spine, the prevailing wisdom for decades has been that increases in physical loading of the spine are beneficial in rehabilitation and exercise, as mediating growth of stronger muscles, tendons, and bones in the spine. Most degenerative vertebral changes are proliferative and adaptive, and serve to maintain spinal function. In spine pathophysiology, increases in load on the spine have been shown to be associated with adaptive changes, analogous to the remodeling of connective tissues in bones in accordance with Wolff's law (see Frost, H. M., The Angle Orthodontist 1994, 64:175-188). Thus, the field has been characterized by a remarkable lack of certitude, and by widely exhibited ambivalence, as to the causal underpinnings, progression, and appropriate therapeutic interventions for structural spine pain.

In this context, one possible reason for the insignificant progress in understanding structural spine pain to date could be that the spine research field has adopted the misdirected notion of pain association with proliferative disc degeneration, and has failed thus far to identify significant proteins and inflammatory genes that are associated with structural spine pain, despite the fact that heredity is known to play an important role in disc degeneration and the incidence of back pain. More specifically, the shape-determining genes associated with vertebral conformation and spinal morphology have received lesser interest, although holding potential for identification of individuals at risk of progressive structural spinal pain.

The use of axial area size in accordance with the present disclosure provides an approach that surmounts these deficiencies of research findings and understanding to date in the efforts to address structural spine pain.

The approach of the present disclosure underscores the shortcomings of the past attempts to identify structural spine pain as a “degenerative disc disease” (DDD), when not all structural spine pain has a degenerative etiology, not all structural spine pain has a nexus with disc characteristics or function, and not all structural spine pain can be characterized as associated with disease. Except for traumatic lumbar spine, the spinal “instability” is not based on measuring, but instead may reflect narrowed intervertebral disc height and belongs to idiopathic back pain. (Videman, T., Battié, M. C., et al., Spine J., 2014, 14:469-78).

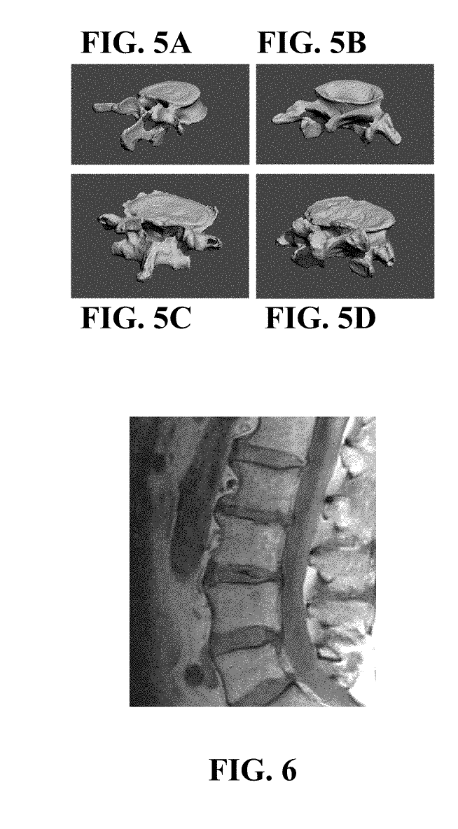

In one aspect, the disclosure relates to a computer-implemented method of quantitatively determining susceptibility to structural spine pain in a vertebrate subject, said method comprising: generating an image, e.g., a magnetic resonance imaging (MRI) image, of a spinal vertebral/cartilaginous endplate locus (for example, upper and lower areas, and 3 intermediate axial areas of the vertebra, so that the axial area of the vertebra may be determined as the mean of such 5 axial areas); determining by computer-implemented determination from data of the image, a proxy mean axial vertebra locus (FIG. 5A-D); determining by computer-implemented determination from the proxy endplate area, a spinal pressure loading value based on body weight of the vertebrate subject; and generating by computer-implemented generation from the spinal pressure loading value, a prognostic characterization of the susceptibility of the vertebrate subject to structural spine pain.

The endplate area size and shape can be generated from routine axial disc imaging, e.g., MRI imaging, to identify disc areas and sagittal diameter of the disc and endplate diameter for determining a “proxy” endplate area, as “proxy” endplate area=disc area×(endplate diameter/disc diameter).

The endplate area size and shape can be generated from routine axial disc imaging, e.g., MRI imaging, to identify disc areas and sagittal diameter of the disc and endplate diameter for determining a “proxy” endplate area, as “proxy” endplate area=disc area×(endplate diameter/disc diameter).

Such method may comprise generating one or more, e.g., 3, values of the spinal pressure loading for each of multiple axial vertebrate loci, e.g., spinal lumbar region loci, based on the mean area determination for the vertebra. It is noted in this respect that the shape is the same at the disc and neighboring vertebra.

In the above-described method, the image may comprise an axial image. Multiple images may be comprised in such image.

The image in the above-described method may be generated by an MRI imaging apparatus comprising an MRI image scanner generating the data of the MRI image in digitized form.

In various embodiments of the above-described method, the proxy endplate area may be determined by calculation of a product of disc area of a disc at the axial vertebra locus and a ratio of vertebral endplate diameter to disc diameter at the axial vertebra locus.

The above-described method in various embodiments may be carried out with the spinal pressure loading value being determined by calculation of the ratio of body weight of the vertebrate subject to the proxy endplate area.

In various embodiments of the above-described method, the proxy endplate area may be determined by calculation of a product of disc area of a disc at the axial vertebra locus and a ratio of vertebral endplate diameter to disc diameter at the axial vertebra locus, and the spinal pressure loading value may be determined by calculation of the ratio of body weight of the vertebrate subject to the axial proxy endplate area, optionally as age-adjusted for the vertebrate subject.

The method broadly described above may further comprise at least one of prescribing and conducting of therapeutic intervention comprising treatment of the patient, based on the prognostic characterization of the susceptibility of the vertebrate subject to structural spine pain.

The vertebrate subject in the above-described method may comprise a human subject, or alternatively a non-human animal subject.

The method as variously described above may be conducted, wherein the operation of generating by computer-implemented generation from the spinal pressure loading value, a prognostic characterization of the susceptibility of the vertebrate subject to structural spine pain, may comprise generating such prognostic characterization in a report comprising at least one of: (i) the vertebrate subject's history of structural spine pain, (ii) correlations of variance of prognosed structural spine pain with future weight of the vertebrate subject, (iii) dietary recommendations, and (iv) avoiding sitting forward flexion. The dietary and/or lifestyle recommendations may be directed to recommendations for avoidance of rapid weight gain or recommendations for minimization of weight gain, and/or other weight gain-opposing lifestyle choices.

The disclosure in another aspect relates to a non-transitory computer-readable storage medium storing instructions executable by a computer system to prognostically characterize susceptibility of the vertebrate subject to structural spine pain, the non-transitory computer-readable storage medium storing instructions to conduct the method as variously described above, or as otherwise described herein.

A further aspect of the disclosure relates to an imaging and computational system for determining susceptibility of a vertebrate subject to structural spine pain, such system comprising: an imaging apparatus configured to generate an image of a axial vertebra locus of the vertebrate subject; and a computer or other processor programmably configured to determine from data of the image generated by the imaging apparatus, a proxy endplate area of the axial vertebra locus, to determine from the proxy endplate area a spinal pressure loading value based on body weight of the vertebrate subject, and to responsively generate from the spinal pressure loading value, a prognostic characterization of the susceptibility of the vertebrate subject to structural spine pain. The imaging may comprise MRI imaging and the imaging apparatus may comprise an MRI apparatus, in various embodiments.

The above-described system may further comprise a database arranged to receive imaging data from the imaging apparatus and computational data from the computer or other processor, for storage in the database, with the computer or other processor being configured to access the database and responsively generate the prognostic characterization of the susceptibility of the vertebrate subject to structural spine pain. The database in various embodiments may contain historical spinal pressure loading values of the vertebrate subject, and/or spinal pressure loading values of a vertebrate subject population, or a selected cohort or sub-population thereof. The database in other embodiments may contain data concerning frequency and severity of structural spine pain events of the vertebrate subject population, or a selected cohort or sub-population thereof.

The system broadly described above may be constituted with the imaging apparatus configured to generate axial images besides of the axial vertebra locus of the vertebrate subject, of axial slices of the whole vertebra height providing accurate axial vertebra area.

In various embodiments, the system broadly described above may be constituted with the computer or other processor operatively linked in communication with a digital communications network, e.g., a local area network, a wide area network, an Internet, or other digital communications network.

The disclosure relates in another aspect to a method of quantitatively determining susceptibility to structural spine pain of a human patient, comprising operating a digital computational apparatus in accordance with a programmed set of instructions to (i) determine from imaging data of an imaged axial vertebra locus and weight of the human patient, a spinal pressure at the endplate locus, and (ii) generate from the determined spinal pressure at the endplate locus a prognostic characterization of susceptibility of the human patient to structural spine pain.

In considering the weight/axial area ratios in the methodologies of the present disclosure, the ratios may be age-adjusted. Measurement of axial areas, although disclosed primarily herein in connection with use of magnetic resonance imaging techniques, since MRI is a most generally utilized tool related to back pain, may also be carried out with any suitable apparatus other than MRI that is effective to image the spine. The applications of the methodologies of the present disclosure although discussed mainly herein in reference to prognostic and therapeutic assessments, also have utility in preventive care, e.g., in communicating warnings of prospective consequences of weight gain. As used herein, the term “patient” is not intended to be construed as implying that a subject individual has a disease condition, as opposed to a physiological condition of actual experience of or potential susceptibility to back pain.

Considering the basic anatomy of the spine, the intervertebral discs are located between upper and lower vertebra of the spine, and when healthy, they function to provide flexibility to the spine and to resist spinal compression, absorbing impacts to the spine that occur with activities conducted on a daily basis. Structurally, the disc is comprised of an outer area known as the annulus fibrosus (referred to herein as simply “annulus”) which surrounds an inner area known as the nucleus pulposus (referred to herein as simply “nucleus”). The annulus of a disc is comprised of several layers of fibrocartilage that, when healthy, strongly contain the nucleus. The nucleus is an arrangement of loose fibers suspended in glycosaminoglycan gel. The healthy disc does not have nerves except surrounding ligament, but during degeneration the annulus can break to form an open canal that allows blood vessels and nerves to grow in the disc but vertebra has blood veins and is innervated and it has some elasticity which decreases with aging.

With disc degeneration, the fibrocartilage of the annulus can break down, developing tears and/or decreased thickness. Assessment and identification of such breakdown is essential to diagnosis of disc degeneration. The axial lumbar spine areas are very similar but their shape changes slowly from roundish L1 vertebra to oval L5 area vertebra. The S1 vertebrae receive support from the pelvis. The structural core that is relevant to structural spine pain is the relative stable size of the axial area, although such area can slowly increase due to adaptation to increasing load. Ultimately, it is the body weight that controls most the pressure and specifically the speed of weight increase and how physiological adaptation is able to increase axial area, as discussed hereinafter in connection with FIG. 6.

The present disclosure recognizes that structural spine pain derives from pressure forces on innervated structures from inside of the lumbar spine without the necessity of structural pathology being involved. In other words, structural spine pain does not fit the parameters of classic illness or disease. The weakness in the studies that have been conducted to date have included the small size of study samples. In contrast, the study on which the present disclosure is based includes one of the largest study samples, over a longitudinal term in excess of 15 years, with repeated magnetic resonance imaging, accurate measurements, and in-depth pain interviews. Due to its size, the study on which the present disclosure is based represents a stable random population and highly reliable quantitative MRI measures. Routine magnetic resonance imaging in some respects is a “rough” tool in investigating macroscopic structures and their actual changes with time, but manual segmenting of endplate borders may be usefully employed for quantitative characterization in the case of uneven endplate areas of severely degenerated spines.

Concerning the study on which the present disclosure is based, among the idiopathic lower back pain questions ask of the study participants, questions covering a prior period of 12 months, both at baseline inception of the study and at follow-up, provide highly reliable data. This provided reliable characterization of back pain with respect to frequency and severity, and prognostic capability. It was indeed surprising that a simple numeric ratio between body weight and vertebra area had a strong back pain predictive capability. The association between disc degeneration measures and weight/proxy vertebra area values was found to be low, underscoring the innovative character of the approach of the present disclosure.

The approach of the present disclosure utilizes the pressure on lumbar spine vertebrae as a selected determinative variable that is “stable” (compared to the disc). Such approach recognizes that increasing continuous pressure on the axial area of the spine increases the pressured area and produces higher areas and decreased pressure, consistent with the observation that the lower lumbar spine is commonly more adaptively degenerated than upper levels of the spine, due to increasing body weight around the navel region. The approach of the present disclosure thus achieves a significant advance over alternative physiological approaches. For example, previous studies have not identified significant protein and inflammatory genes associated with back pain, in spite of the dominant role of heredity in disc degeneration and back pain.

The use of the ratio of selected vertebra endplate and disc regions may be achieved by lumbar spine morphometric measurements of distances, areas of the boundaries of vertebrae and discs in selected MR image sequences, and techniques such as proton density (PD) that show clearly the boundaries between bone, cartilage, annulus and ligaments and soft tissues covering the spine.

The present disclosure recognizes that the magnitude of pressure on the axial area of the spine is a primary constitutional risk factor for structural spine pain, and that the ratio of the body weight of a patient to the proxy vertebra axial area provides values that identify structural spine pain and predict structural spine pain as much as 15 years, and more, in the future. Body weight is of course partially behavioral in character, as well as having constitutional and external components as determinative factors.

Thus, the morphological differences in vertebra may be associated with vertebral endplate structures of substantially varying morphological character and surface roughness, and since the vertebral endplate acts as a template for the cartilaginous endplate structure, the above-discussed proxy endplate area affords an approach that is quantitatively reliable and reproducible.

The methodologies of the present disclosure variously include assessment, diagnosis, and prognosis of structural spine pain, as well as determinations of risk values of structural spine pain.

In these methodologies, a magnetic resonance image of the axial plane of the spine at the axial vertebra area is obtained in digital form, and each of the proxy, or adjusted, endplate areas are determined by lumbar spine level, with subsequent calculation of the pressure loading values of the spine at such loci. The pressure loading value will be employed as a risk value representing structural spine pain. The calculation of the adjusted endplate area of structural spine pain is carried out using a computer-implemented analysis program comprising appertaining algorithm(s) for such calculation. An accurate slice of the axial endplate and measure of the area of the endplate is carried out. In analyzing earlier standard magnetic resonance images, mid axial disc area provides a proxy enabling the endplate area to be determined using mid sagittal disc and endplate diameters. The ratio of the patient's body weight to the endplate area provides a measure of pressure at the endplate and is an objective indicator risk of back pain. A computer-implemented analysis program may be employed to quantitate the axial endplate area, utilizing axial and sagittal images as appropriate to correspondingly determine the proxy endplate area, and to provide correlative computations of the pressure loading at the specific locus of the spine of the patient, using body weight of the patient. The body weight measurement may be made contemporaneously with the magnetic resonance imaging of the spine of the patient.

In various embodiments, the computer-implemented analysis program may be configured to determine the dimensional characteristics of a spinal endplate region from a magnetic resonance image, to calculate a corresponding specific proxy area, and to calculate the pressure loading value at each relevant spinal endplate locus. The individual spinal pressure loading values at multiple spinal endplate loci can then be individually assessed, or can aggregately be used to determine mean values of endplate proxy area values determined for incremental imaging slices in the vicinity of the endplate, FIG. 4. The analysis program may be employed to analyze earlier standard magnetic resonance images at a mid-axial disc area to provide a proxy of determining endplate area using mid sagittal disc and endplate diameters.

Thus, a computer-implemented analysis program may be employed comprising relevant algorithm(s) for carrying out the spinal pressure loading value(s).

Although it generally is desirable to fully automate the determination of the spinal pressure loading value utilizing computational hardware configured to carry out processing instructions in a corresponding software program for such determination, the present disclosure also contemplates computational analysis of endplate or other vertebral sections that are manually segmented, e.g., by manual tracing of endplate and/or other spinal structure regions that then constitute a demarcated area that is computationally determined by the software program. The software program in this respect may be specially designed to automatically recognize the areas and distances of interest, and to obtain measures within the identified areas. Alternatively, a software program may be utilized that accepts the user defined areas and distances.

The endplate areas can be counted based on disc areas and using sagittal diameter of the disc and endplate diameter and the software provides these diameters after the sagittal vertebrae and discs areas are segmented. The disc size was not used because disc area increases with aging but the area begins to decrease later in the life, however the ‘copy’ of the shape is same in relation to ‘thatching’ with the vertebra's area. This method is used when using the current imaging routines in sagittal MRI settings, where the mid image is mainly used in diagnostics, but setting the middle axial image though the endplate area with the software providing the area size, optionally as age-adjusted for the vertebrate subject. It is probable that soon there will be available accurate methods, such as ultrasound and low radiation computer tomograms as example, which provide directly whole vertebrae's all measures, however they cannot measure earlier MRI images, which could identify the risk factor among those with suspected spine pressure pain without a new MRI imaging.

The present disclosure therefore achieves a substantial advance in the art, by the discovery that structural spine pain is spinal pressure pain, with such discovery correspondingly enabling rational prevention and treatment, such as weight control and sitting and working positions, to be implemented to minimize the frequency and severity of such pain.

The simple determination of spinal pressure loading values, as a ratio body weight to a proxy axial area at the endplate region of the vertebra, provides a diagnostic tool for identifying and quantifying risk factors of back pain. The spinal pressure loading values can be used by persons including family physicians, radiologists, orthopedics, neurosurgeons, sports medicine doctors, occupational doctors, physiotherapists, chiropractors, and researchers and are desirably added in spine MRI software.

The spinal pressure loading value methodology of the present disclosure represents a morphometric risk factor identification technique that can predict back pain at least up to 15 years in advance and potentially even from childhood. The predictive character of the methodology of the present disclosure embodies an extraordinary simplicity that is readily implementable by use of magnetic resonance imaging scanners programmed in accordance with the present disclosure to determine the proxy axial area of the spinal endplate, and to generate an output correlative of such proxy axial area, wherein the MRI programmed scanner output, or output from other imaging apparatus, is transmitted to a processor configured to calculate the spinal pressure loading value for such axial area. The specific spinal pressure loading value that is thus determined can then be correlated with data in a database accessible to the processor, to computationally determine a prognostic value at a longitudinal time determined in relation to the database values with which the spinal pressure loading value is correlated. As discussed above, the spinal pressure loading value can be utilized for prognostication of structural spine pain at a future time within the temporal scope of the database. The database may be resident in a memory coupled in signal processing relationship to the processor, so that database information is computationally available to the processor. The processor itself may be of any suitable type, and may comprise a microprocessor, programmable logic unit, special purpose programmed computer, or other suitable processor.

Consistent with the foregoing, a further aspect of the disclosure relates to a non-transitory computer-readable storage medium storing instructions executable by a computer system to automatically quantify the risk value of the back pain from body weight data of a patient, and proxy axial vertebra area data of the patient determined from imaging of a patient's spine.

The present disclosure in other aspects relates to systems, non-transitory computer-readable media, and methods for assessing spinal axial areas measures using spinal morphometrics of endplate edges and discs in a quantitative manner using magnetic resonance imaging (MRI) data, comprising computation of spinal pressure loading value(s) from endplate axial area edges by determining the ratio of body weight to proxy endplate area in the lumbar spine.

Such systems, non-transitory computer-readable media, and methods enable a measure of lumbar spinal health to be generated, wherein the measure is calculated using the ratio from morphometric measurements of lumbar spine structures or regions and human body weight. The morphometric measurements of lumbar spine structures or regions can be obtained either through manual segmenting or automated through programming of image analysis software. Spinal measurements in accordance with the present disclosure are readily generated from MRI scanner data for the spinal vertebral locus or loci of interest. Multiple spinal vertebral loci may be employed to accurately delineate particular regions of particular susceptibility (risk factor) to structural spine pain, which in turn can assist in interventional preventions, treatments and therapies.

The present disclosure in one aspect contemplates a methodology in which a clinician or other medical personnel obtains selected images, e.g., MRI images, of a lumbar spinal vertebral locus of a patient, which images are then digitized, and in which the clinician or other medical and health and wellness personnel selects areas of the digitized images for input of corresponding area selections to a computer or other processor, for computational determination by the computer or other processor of an axial endplate area of at least one lumbar spinal vertebral locus of the patient, and computational determination by the computer or other processor of a ratio of body weight of the patient to the axial endplate area of the lumbar spinal vertebral locus of the patient, as a spinal pressure loading value of the patient at the lumbar spinal vertebral locus, and outputting by the computer or other processor to the clinician or other medical or health and wellness personnel of the spinal pressure loading value, and at least one of prescription and conduct by the clinician of therapeutic intervention comprising treatment of the patient, based on the outputted spinal pressure loading (risk) value of the patient.

In other aspects, a variation of the foregoing methodology may be carried out, in which the clinician or other medical personnel traces, e.g., by manual tracing, areas of the digitized image for input of the corresponding area selections to the computer or other processor.

In still other aspects, a variation of the foregoing methodology may be carried out, in which the area selections for input to the computer or other processor are predetermined, and inputted by an imaging apparatus to the computer or other processor, e.g., at the time the selected MRI images are generated by the imaging apparatus.

In carrying out the foregoing methodology, the corresponding imaging and computational system may be interconnected in signal transmission relationship, so that imaging data are transmitted by the imaging apparatus to the computer or other processor contemporaneously with image generation by the imaging apparatus, or at a subsequent point in time. As previously described, the imaging and computational system may be constituted with a database, e.g., in a memory component of the computational apparatus, so that imaging and computational data are transmitted to the database for storage, and with the computer or other processor of the computational apparatus being configured to access the database and responsively generate, from newly generated patient data and archival data in the database, an output indicative of spinal health of the patient comprising a prognosticated susceptibility and severity of structural spine pain at a selected future time or times.

In various embodiments, the archival data in the database may comprise historical structural spine pain spinal health data generated by the methodology of the present disclosure for the same patient at a prior time or times, whereby the output comprises a longitudinal report of time-varying structural spine pain spinal health of the patient and prognosticated susceptibility and severity of structural spine pain at a selected future time or times. In other embodiments, the archival data in the database may comprise structural spine pain spinal health data for a patient population generated by the methodology of the present disclosure, whereby the output comprises a comparative report of structural spine pain spinal health of the patient in relation to the patient population, or a selected cohort sub-population thereof. In still other embodiments, the archival data in the database may comprise historical structural spine pain spinal health data generated by the methodology of the present disclosure for the same patient at a prior time or times, wherein the computer or other processor of the computational apparatus is configured to generate a prognostic report for the patient comprising prognostic spinal health of the patient at a future time or times, based on the progression or character of prior structural spine pain spinal health data generated by the methodology of the present disclosure. Such prognostic report may alternatively be generated based on archival data in the database comprising spinal health data for a patient population generated by the methodology of the present disclosure, whereby the report is based on progression of spinal health for such patient population, or a selected cohort sub-population thereof. In such manner, risk factors can be generated for the patient, in respect of structural spine pain.

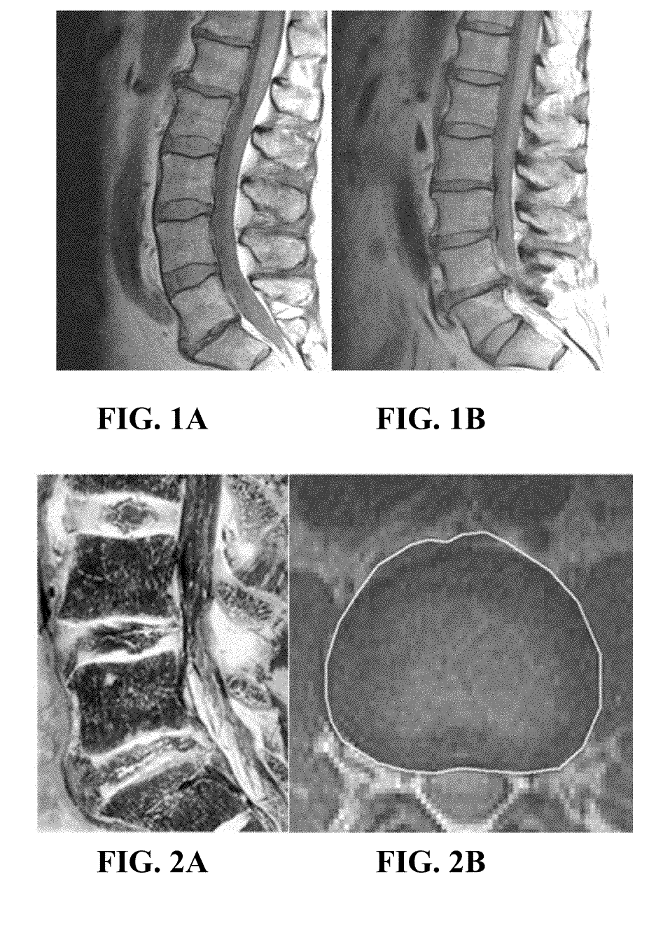

Referring now to the drawings, FIG. 1A and FIG. 1B are MRI images of respective spines exhibiting degeneration. In FIG. 1A, the highest two and lowest discs are severely degenerated. In FIG. 1B, the lowest disc is healthy and the three above are degenerated, which is a common degenerated state.

FIG. 2A is a cryomicrotome figure showing three lumbar spine levels, in which the upper disc is healthy, and the lower disc is severely degenerated. The darker areas in the disc are gel. FIG. 2B is an axial disc image on which a white line has been manually traced along the disc's outer edge.

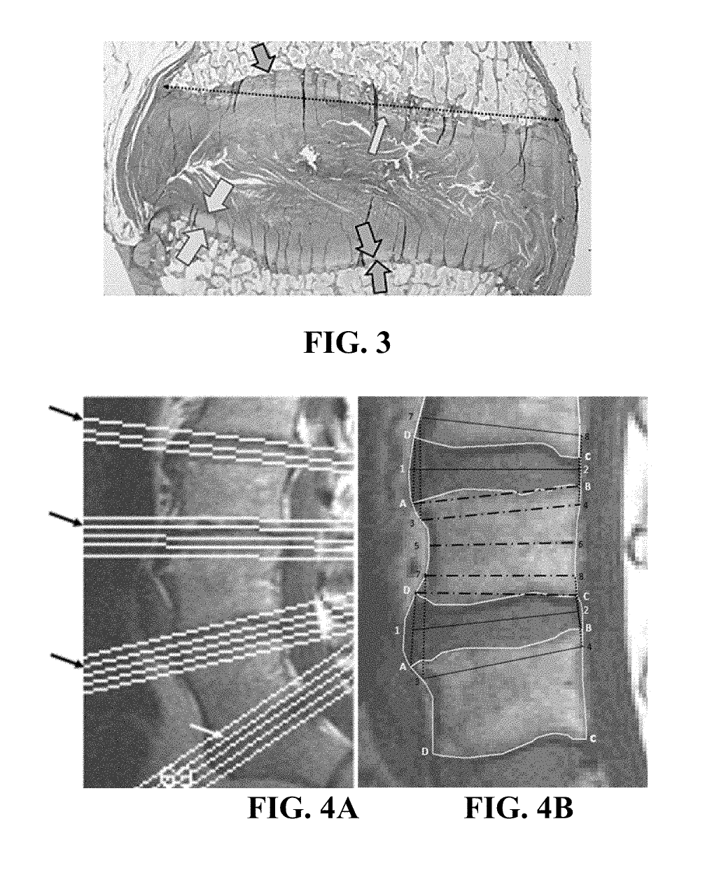

FIG. 3 is a sagittal histologic image of an inter-vertebral disc on which the superimposed dotted line represents the axial length between bony endplate ends, wherein the bony endplate ends represent clear stable points of with pressure area. The darker arrows point to bony endplates and the lighter arrows point to cartilaginous endplates.

FIGS. 4A and 4B are sagittal MRI images, illustrating how vertebra endplate area is measured. In FIG. 4A, the mid-sagittal vertebra image is supplemented by 4 arrows pointing to axial slices that are close to the endpoint areas, however these produce errors due to disc height variation. In FIG. 4B, the mean of the 5 dashed lines (shown as AB, 34, 56, 78, DC) is used as the mid width of that vertebra. The upper linear horizontal black line 1-2 in each instance is used as the mean disc diameter.

The sagittal MR image in FIG. 4A illustrates typical axial disc areas. The figure shows a middle axial ‘slicing’ of 4 sagittal discs as is a routine practice in spine MRI imaging. The 4 arrows pointing to axial FIG. 3 is a sagittal histologic image of an intervertebral disc on which the superimposed dotted line represents the axial (transverse) dimension between lateral extremities of the bony endplate, wherein the bony endplate ends represent clear stable regions of width pressure area. The darker arrows point to bony endplates and the lighter arrows point to cartilaginous endplates.

FIG. 4B is a sagittal vertebra magnetic resonance image in which the mean of the 5 black dotted lines, A-B, 3-4, 5-6, 7-8, and D-C, is used as the mid-width of that vertebra; with the upper and lower linear horizontal black lines 1-2 of disc mid-width showing the mean axial disc area to be counted.

The vertebrae in FIGS. 5A-5D were imaged by computer tomography (a microCT scanner) which enabled visualization of the internal structure and measurement of areas and distances. The imaging required for the methodologies of the present disclosure, including axial area measurements, can be done with computer tomography, or alternatively, with sonography utilizing ultrasound apparatus, or other radiation-based or non-radiation techniques, instruments, and apparatus. It will be recognized that any reference herein to MRI imaging may alternatively be specified generally by imaging, utilizing any suitable imaging and/or visualization technologies and approaches.

FIG. 6 is a magnetic resonance image of a middle lumbar spine area of a MR image at 15 years' follow-up and it shows exceptionally large anteriorly increased (to the left) vertebra endplates of the male subject, who had lost 17 kg weight. Although individual cases can be random, this subject's risk value for structural spinal pain had decreased from 5.8 to 4.5 in lower two vertebrae and from 5.3 to 3.9 in two upper vertebrae (and at the baseline interview he did not have back pain).

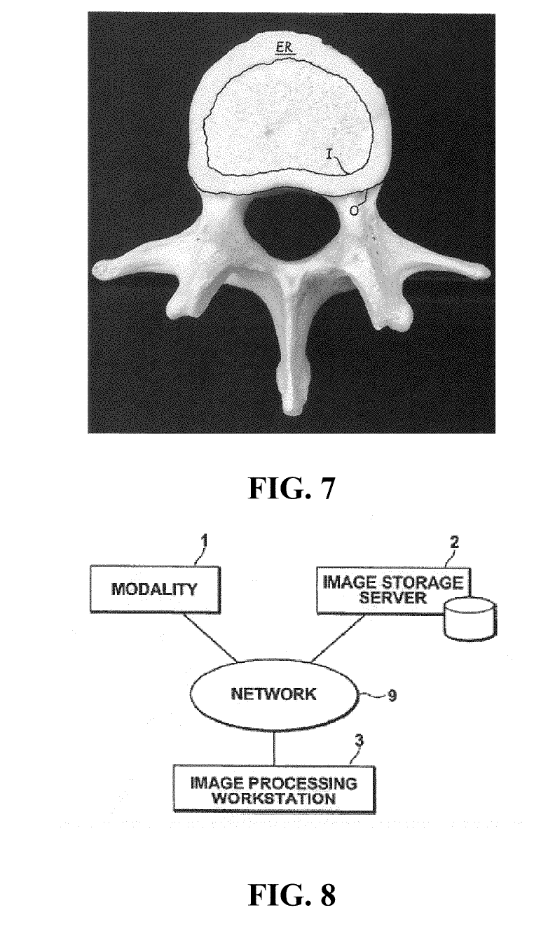

FIG. 7 is a top plan view of a spinal vertebra, showing the epiphyseal ring (ER) of the vertebral body, as defined by the inner tracing line (I) and the outer tracing line (O).

FIG. 8 is a schematic representation of a magnetic resonance image processing system according to one embodiment of the present disclosure. The disclosure contemplates imaging and computational systems configured to perform the various aspects and embodiments of the methodology described above, as well as non-transitory computer-readable storage media storing instructions executable by a computer system, which may comprise a computer or other processor, to quantify structural spine pain spinal health of a patient by any of the methodology aspects and embodiments described above.

As depicted in FIG. 8, the system includes modality 1, image storage server 2, and image processing workstation 3 communicatively linked to each other via network 9.

The modality 1 is an apparatus for magnetic resonance imaging of the spine of a vertebrate subject, e.g., a human or other vertebrate animal subject, to generate imaging data representing the spinal regions and outputting the image data. The modality 1 may include any suitable MRI equipment and assemblies. The imaging data may be outputted by addition of auxiliary information defined by DICOM (Digital Imaging and Communications in Medicine) standard as image information.

The image storage server 2 comprises a computer or other processor that is configured to store and manage in an imaging database the imaging data that is obtained by modality 1 as well as the proxy endplate area data and the spinal pressure loading value data that are generated by the image data processing workstation 3. The image storage server 2 may be configured in any suitable manner, and may comprise high capacity external storage media and devices and database management software, e.g., Object Relational Database (ORDB) management software.

The image processing workstation 3 is configured to generate the proxy endplate area data and the spinal pressure loading value data as well as historical and prognostic reports including such spinal pressure loading value data and corresponding assessments of future susceptibility and severity of structural spine pain of the patient. Such assessments of future susceptibility and severity of structural spine pain of the patient may include general population, cohort, and/or sub-cohort data derived from the database in the image storage server. The image processing workstation 3 may be programmatically arranged with appropriate operating system and application software to process the imaging data obtained by modality 1, as accessed through the network 9, and to responsively generate the spinal health report so the patient and other output. Such reports and outputs may be transmitted via the network 9 to the image storage server 2, and may be displayed on the display of the image processing workstation, or otherwise exported from the system in appropriate form.

The image processing workstation 3 may also be programmatically arranged to process imaging data that is stored in and accessed from image storage server 2, to provide appropriate output, e.g., longitudinal monitoring of structural spine pain, or comparison of current condition data, as obtained from the modality 1, with a last-determined or time-averaged measure of structural spine pain events and pain severity, as obtained from image storage server 2.

The image processing workstation 3 may therefore be configured to enable a medical clinician to utilize the workstation for displaying structural spine pain reports, as well as correlative and ancillary output, for retrieving historical imaging data from the image storage server, and for information transmission of structural spine pain reports and associated output to recipient computers and clinicians via the network 9. For such purpose, the network 9 may be of any suitable character, and may comprise a local area network (LAN), global network such as a global Internet, and/or other network of a wired, wireless, or other character.

The image processing workstation 3 is appropriately configured for the functions described above, and may for example comprise a hardware configuration including a CPU, main memory, auxiliary memory, I/O interface, communication interface, input device (mouse, keyboard, and the like), display monitor, data bus, and the like, with a suitable operating system installed thereon. The imaging data processing conducted by the image processing workstation 3 may be carried out by execution of a spinal lumbar region analysis program performing a methodology of the present disclosure to generate a structural spine pain report and optionally correlative and ancillary data, based on input imaging data from modality 1 and/or image storage server 2.

Such spinal lumbar region analysis program may be installed or accessed from a suitable recording medium, such as a CD-ROM or the like, that is non-transitory and computer readable in character. The program may alternatively be downloaded and installed on the workstation from a storage device of a server linked to network 9, e.g., the image storage server 2, or other server accessible to the network 9, or by other independent network to which the workstation is communicationally linked. As a further alternative, the program may be partially or wholly embodied in firmware, or in other non-transitory computer-readable medium.

The imaging data storage format and communications between each component of the FIG. 8 system via the network 9 may be based on any suitable communications protocol, e.g., the DICOM protocol.

It will be recognized from the foregoing that the systems, computer-readable media, and methods described herein achieve a substantial advance in the art for assessing historical, current, and/or future structural spine pain condition of a patient in a quantitative manner using magnetic resonance imaging (e.g., MRI) data, and that such systems, media, and methods may be implemented in a wide variety of applications. Such applications may include, for example, research applications, such as investigating determinants of structural spine pain and pathology and their progression, as well as screening for risk of structural spine pain, diagnosing spinal pathology, prognosing disc conditions and spinal health, monitoring and assessing effects of therapeutic interventions in spinal and disc treatments, and the like.

The features and advantages of the approach of the present disclosure are more fully illustrated by the following Example.

### Example—Finnish Twin Cohort Study

The study population sample for this study included participants selected from 232 men initially recruited in a twin spine study, which were drawn from the population-based Finnish twin cohort born in Finland before 1958 and still alive in 1975. The monozygotic subjects in the twin spine study have been shown to be highly representative of the Finnish twin cohort, which in turn is representative of the male Finnish population. See Battié, M. C., Videman, T., et al., Spine, 1995, 20:2601-12. An extensive baseline interview included questions about the frequency and impact of lower back pain and MRI scans were obtained. The lower back pain questions covered the prior 12 months both at baseline and follow-up, which provided the most reliable data. This provided reliable back pain data with respect to frequencies and severity. Fifteen years later, men who were still living were invited for follow-up. MRI images of acceptable image quality and pain outcomes were available for 108 men. The mean age at the time of the final follow-up was 63 years (range 50-79 years). All subjects received written information about the study procedures before participation and the study protocols were reviewed and approved by the Ethical Committees of the Department of Public Health at the University of Helsinki and the University of Alberta.

The baseline data and 15-year follow-up data are set out in Table 1 below (SD=standard deviation).

TABLE 1

| | Baseline | 15-year Follow-up |
|---|---|---|
| 108 men | mean (SD) | mean (SD) |
| Age | 47.2 (7.8) | 63.1 (8.1) |
| Weight (kg) | 79.5 (11.2) | 81.5 (12.0) |
| Height (cm) | 174.7 (7.0) | 174.4 (7.4) |

Statistical methods for the study are described below. The spinal morphometrics used to search for associations with reported back pain in prior year included: vertebra height and width, disc height and width, axial disc area, the disc volume proxy, body weight/axial disc area, body weight/proxy vertebra area, and body weight/vertebra area. The means of L2-S1 spine and L2-L4 and L4-S1 comprised the upper and lower lumbar levels, respectively.

Two sets of models were used to predict each primary and secondary structural spine pain measure at 15 years: one set using baseline spinal morphometrics and one using the change from baseline. Ordinal logistic models were controlled for baseline age, height, and weight, and the baseline structural spine pain measure, except in the case of the three baseline predictors that included weight, where weight was not included as an additional covariate. Models were checked for fit, and Poisson regression models were run to confirm statistical significance. In addition, percent change from baseline in each morphometric measure was assessed in confirmatory analyses. All variables with p-values less than 0.05 were candidates for a multivariate model. In all models, the correlation between twins was accounted for in the variance estimates. With larger populations and both genders the prevalence of structural spine pain is more accurate and what are the typical signs in structural spine pain. After that and has confirmed the role of structural spine pain it is questing the rationality of several current therapies used to treat for idiomatic back pain.

The data generated in the study included the data in Tables 2 and 3 below.

TABLE 2
Spine Morphometrics -15 Year Prediction of structural spine pain.

| | OR (95% CI) | P | OR (95% CI) | P | OR (95% CI) | P | OR (95% CI) | P |
|---|---|---|---|---|---|---|---|---|
| L2-L4 levels | | | | | | | | |
| Disc height | 0.77 (0.54, 1.09) | 0.1408 | 0.87 (0.65, 1.17) | 0.3702 | 1.22 (0.68, 2.17) | 0.5035 | 1.41 (0.88, 2.25) | 0.1550 |
| (millimeters) | | | | | | | | |
| Weight/disc area | 2.14 (1.06, 4.33) | 0.0350 | 1.44 (0.67, 3.10) | 0.3537 | 3.08 (1.45, 6.52) | 0.0033 | 2.98 (1.43, 6.21) | 0.0037 |
| (100*kg/mm²)† | | | | | | | | |
| Weight/vertebra | 1.66 (1.14, 2.41) | 0.0087 | 1.18 (0.75, 1.85) | 0.4684 | 2.33 (1.27, 4.30) | 0.0067 | 1.95 (1.26, 3.00) | 0.0026 |
| area (100*kg/mm²) | | | | | | | | |
| Weight/vertebra | 4.02 (1.24, 13.1) | 0.0208 | 1.74 (0.48, 6.26) | 0.3967 | 7.72 (2.04, 29.2) | 0.0026 | 7.25 (2.21, 23.74) | 0.0011 |
| area + disc area | | | | | | | | |
| (100*kg/mm²) † | | | | | | | | |
| L4-S1 levels | | | | | | | | |
| Disc height | 1.00 (0.83, 12.2) | 0.9626 | 1.02 (0.81, 1.28) | 0.8914 | 0.93 (0.65, 1.34) | 0.7079 | 1.00 (0.68, 1.47) | 0.9974 |
| (millimeters) | | | | | | | | |
| Weight/disc | 2.12 (1.14, 3.93) | 0.0175 | 1.52 (0.78, 2.95) | 0.2219 | 1.59 (0.57, 4.41) | 0.3760 | 1.89 (0.75, 4.73) | 0.1741 |
| area (100*kg/mm²)† | | | | | | | | |
| Weight/vertebra | 1.70 (1.16, 2.50) | 0.0067 | 1.20 (0.75, 1.91) | 0.4545 | 3.00 (1.42, 6.32) | 0.0040 | 2.08 (1.13, 3.84) | 0.0189 |
| area (100*kg/mm²) | | | | | | | | |
| Weight/vertebra | 4.11 (1.43, 11.81) | 0.0086 | 1.94 (0.58, 6.48) | 0.2801 | 4.05 (0.57, 28.88) | 0.1626 | 4.53 (0.90, 22.86) | 0.0673 |
| area + disc area | | | | | | | | |
| (100*kg/mm²) † | | | | | | | | |

†Baseline weight is not a covariate in the Baseline models.

TABLE 3

| | Baseline pressure value and Baseline Pain | | | | 15 year pressure value and 15 year Pain | | | |
|---|---|---|---|---|---|---|---|---|
| | Back pain frequency | | Back pain disability | | Back pain frequency | | Back pain disability | |
| Lx | OR (95% CI) | p-value | OR (95% CI) | p-value | OR (95% CI) | p-value | OR (95% CI) | p-value |
| L2 | 0.99 (0.65, 1.52) | 0.9641 | 1.05 (0.70, 1.57) | 0.8161 | 1.86 (1.41, 2.46) | 0.0000 | 1.35 (1.01, 1.81) | 0.0445 |
| L3 | 0.88 (0.58, 1.34) | 0.5564 | 0.86 (0.58 1.28) | 0.4608 | 1.76 (1.25, 2.48) | 0.0012 | 1.31 (0.97, 1.78) | 0.0778 |
| L4 | 0.89 (0.56, 1.42) | 0.6238 | 0.78 (0.51, 1.17) | 0.2290 | 1.89 (1.30, 2.76) | 0.0009 | 1.28 (0.92, 1.77) | 0.1435 |
| L5 | 0.97 (0.64, 1.47) | 0.8770 | 0.77 (0.55, 1.08) | 0.1317 | 1.92 (1.20, 3.09) | 0.0070 | 1.34 (0.91, 1.96) | 0.1387 |
| | Baseline pressure value and 15 year Pain | | | | Baseline pressure value and 15 year Pain* | | | |
| | Back pain frequency | | Back pain disability | | Back pain frequency | | Back pain disability | |
| | OR (95% CI) | p-value | OR (95% CI) | p-value | OR (95% CI) | p-value | OR (95% CI) | p-value |
| L2 | 1.60 (1.06, 2.42) | 0.0266 | 1.20 (0.79, 1.83) | 0.3911 | 1.68 (1.18, 2.40) | 0.0041 | 1.16 (0.74, 1.82) | 0.5176 |
| L3 | 1.41 (0.93, 2.13) | 0.1078 | 1.15 (0.76, 1.72) | 0.5092 | 1.53 (1.03, 2.28) | 0.0358 | 1.13 (0.73, 1.75) | 0.5801 |
| L4 | 1.57 (1.00, 2.47) | 0.0486 | 1.11 (0.71, 1.74) | 0.6531 | 1.72 (1.16, 2.57) | 0.0073 | 1.12 (0.70, 1.81) | 0.6332 |
| L5 | 1.48 (0.96, 2.28) | 0.0792 | 1.14 (0.76, 1.70) | 0.5357 | 1.56 (1.09, 2.23) | 0.0159 | 1.21 (0.79, 1.86) | 0.3879 |

*also controlling for baseline back pain frequency or disability, respectively.

The data in Table 2 show back pain at 15 years predicted by models with baseline spinal morphometric standby models with change from baseline in spinal morphometrics, controlling for baseline age, height, and weight, and baseline back pain frequency or disability, respectively. The OR values are given for 1 point on a 7-point back pain scale and for one point on a 4-point back pain disability scale. The pressure value of change from baseline based on sum of the vertebra and disk axial areas are very high in the upper lumbar region but not in the lower lumbar spine region, due to shrinkage of the disc in the more degenerated disc areas.

Table 3 sets forth the data for the lower four lumbar vertebrae (L2, L3, L4, and L5) as simple predictors, using baseline measures and back pain at 15 years' follow-up. The data show that the approach of the present disclosure is highly effective in more degenerated spine patients.

Thus, the pressure value determined by the ratio of body weight/proxy axial area provides a quantitative measure of identified and predictive back pain, and represents an advance in the understanding of structural spine pain, as fundamentally needed for rational prevention and therapeutic intervention, as variously described herein. In addition to constitutional (morphologic) risk factor analysis, the quantitative measure utilized in the present disclosure also reflects prognostic measures of behavioral risk factor, namely, weight and specifically weight variations (both increases and decreases).

In measuring spine area in accordance with the present disclosure, the axial vertebra area between the disc and vertebra has been selected, because vertebra and the discs' areas can vary significantly in a shorter time but vertebra axial area can increase until old age. The “proxy” axial vertebra area is determined by the equation: “proxy” axial vertebra area=disc area×(axial vertebra diameter/disc diameter), and presumes that the shape of the disc corresponds to the shape of the axial vertebra area. That area can be directly determined from MRI or other images if the axial slice of the spine is adjusted to include endplate corners in the imaging software instructions. The proxy endplate area approach of the present disclosure exhibited less variation between baseline and follow-up data and had the highest correlation with spinal pain. The corresponding pressure loading on the spine at a given vertebral locus is calculated as

spinal pressure loading=body weight/proxy axial area

where the proxy axial area of the specific vertebra is its proxy axial vertebra area determined as described above from the disc area, axial vertebra diameter, and disc diameter. Neither the axial area nor weight alone associate statically significant structural spine pain.

The magnitude of the spinal pressure loading identifies and predicts back pain, e.g., as age-adjusted for the subject. Body weights vary commonly and endplate area can increase, but slowly compared to the rate of increase of body weight.

The capability of the approach of the present disclosure in predicting back pain at least over a period of 15 years (which was the longest follow-up with MRIs) is an exceptionally strong validation of the general methodology described herein. Considering the sample size employed in the Finnish twin study, the exceptionally low p-values and high odds ratios (ORs) of associations between adjusted endplate area pressure and idiopathic pain and exclude chances of random findings. Among all men who had had back pain over the preceding 12 months, 67% of them belonged to the group with the highest risk values and 75% of those with back pain monthly or more often belong to this group with highest risk values. In this population sample 67% had not had back pain during the 12 months before the baseline and during the 12 months before the 15-year follow-up. The axial disc area sizes are continuous from smallest to highest and in middle region the pain varies due to weight variation. There are also those who have high risk value who have not have reported back pain at baseline, but have back pain 15 years later and “critical” values can vary among people (Table 3). Those patients with high risk values do not necessarily exhibit symptoms continuously, since the nature of structural spine pain is that the pain can disappear when lying prone, sitting nonchalantly, or flexed forward and supported with elbow (Wilke, H. J., Neef, P., et al. Spine, 1999:15:755-62). Goff concluded in 1955 that “[t]hese mechanical strains have contributed to many types of low backache as ‘fatigue’” in his study “[p]ostural evolution related to backache” (Goff, C. W. Clin. Orthop.1955:5:8-16). The episodic and sporadic character is explainable in a majority of cases by body weight variation and the speed of such body weight variation, and the extent of the non-sleep time in which a patient is engaged in a sitting as opposed to a standing position. This could explain why persons report that they have never had back pain, but in detailed inquiry practically all have experienced back pain and this difference relates to the perception that many individuals do not consider disturbing feeling along the spine to be back pain, in contrast to local sharp or dull aches that they consider to be characterizable as pain caused from disease. There are of course other unknown back pain causes that may impact a specific individual, but the approach of the present disclosure can explain most structural spine pain, and embodies a substantial advance in the art.

The present disclosure therefore provides systems, computer-readable media, and methods for identification and prediction of structural spine pain.

In a specific aspect, the disclosure relates to computer-implemented method of quantitatively determining susceptibility to structural spine pain in a vertebrate subject, said method comprising: generating or obtaining an image of a spinal external epiphyseal ring of a spinal axial vertebra area locus; determining by computer-implemented determination from data of the image, a proxy endplate area of the spinal axial vertebra area locus; determining by computer-implemented determination from the proxy endplate area, a spinal pressure loading value based on body weight of the vertebrate subject; and generating by computer-implemented generation from the spinal pressure loading value, a prognostic characterization of the susceptibility of the vertebrate subject to structural spine pain.

Such method may be carried out with generation of spinal pressure loading value for each of multiple spinal axial vertebra area loci, e.g., spinal lumbar region loci. The image generated or obtained in such methodology may comprise a multiplicity of axial images, e.g., at top, bottom, and 3 intermediate portions of a vertebra, e.g., in which the image is generated by an imaging apparatus outputting image data in digital form. The proxy endpoint area may be determined by calculation of a product of disc area of a disc at the spinal axial vertebra area locus and a ratio of vertebral endplate diameter to disc diameter at the spinal locus. The spinal pressure loading value may be determined by calculation of the ratio body weight of the vertebrate subject to the proxy endplate area, e.g., as determined by calculation of a product of disc area of a disc at the spinal axial vertebra area locus and a ratio of vertebral endplate diameter to disc diameter at the spinal axial vertebra area locus, wherein the spinal pressure loading value is determined by calculation of the ratio body weight of the vertebrate subject to the proxy endplate area, optionally as age-adjusted for the vertebrate subject.

The above-described method may further comprise at least one of prescribing and conducting of therapeutic intervention comprising treatment of the vertebrate subject, e.g., a human vertebrate subject, based on the prognostic characterization of the susceptibility of the vertebrate subject to structural spine pain.

The above-described method may further comprise generating by computer-implemented generation from the spinal pressure loading value, a prognostic characterization of the susceptibility of the vertebrate subject to structural spine pain, comprises generating said prognostic characterization in a report comprising at least one of: (i) the vertebrate subject's history of structural spine pain, (ii) correlations of variance of prognosed structural spine pain with future weight of the vertebrate subject, (iii) dietary recommendations, and (iv) lifestyle recommendations.

The disclosure also contemplates a non-transitory computer-readable storage medium storing instructions executable by a computer system to prognostically characterize susceptibility of the vertebrate subject to structural spine pain, the non-transitory computer-readable storage medium storing instructions to conduct the method of the disclosure, e.g., as described above in the various embodiments herein.

Further, the disclosure contemplates an imaging and computational system for determining susceptibility of a vertebrate subject to structural spine pain, said system comprising: an imaging apparatus configured to generate an image of a spinal axial vertebra area locus of the vertebrate subject; and a computer or other processor programmably configured to determine from data of the image generated by the imaging apparatus, a proxy endplate area of the spinal axial vertebra area locus, to determine from the proxy endplate area a spinal pressure loading value based on body weight of the vertebrate subject, and to responsively generate from the spinal pressure loading value, a prognostic characterization of the susceptibility of the vertebrate subject to structural spine pain.

Such system may further comprise a database arranged to receive imaging data from the imaging apparatus and computational data from the computer or other processor, for storage in the database, with the computer or other processor being configured to access the database and responsively generate the prognostic characterization of the susceptibility of the vertebrate subject to structural spine pain. The database may contain historical spinal pressure loading values of the vertebrate subject, and/or spinal pressure loading values of a vertebrate subject population, or a selected cohort or sub-population thereof. The database may contain data concerning frequency and severity of structural spine pain events of a vertebrate subject, or vertebrate subject population or selected cohort or sub-population thereof.

The system may in specific embodiments be constituted with the imaging apparatus configured to generate axial images of the spinal axial vertebra area locus of the vertebrate subject. In various embodiments, the computer or other processor of the system may be operatively linked in communication with a digital communications network.

In various implementations, the disclosure contemplates a method of quantitatively determining susceptibility to structural spine pain of a human patient, comprising operating a digital computational apparatus in accordance with a programmed set of instructions to (i) determine from imaging data of an imaged spinal axial vertebra area locus and weight of the human patient, a spinal pressure at the axial vertebra area locus, and (ii) generate from the determined spinal pressure at the axial vertebra area locus a prognostic characterization of susceptibility of the human patient to structural spine pain.

While the disclosure has been set forth herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the disclosure as hereinafter claimed is intended to be broadly construed and interpreted, as including all such variations, modifications and alternative embodiments, within its spirit and scope.

What is claimed is:

1. A computer-implemented method of quantitatively determining susceptibility to structural spine pain in a vertebrate subject, said method comprising:
generating or obtaining an image of a spinal external epiphyseal ring of a spinal axial vertebra area locus;
determining by computer-implemented determination from data of the image, a proxy endplate area of the spinal axial vertebra area locus;
determining by computer-implemented determination from the proxy endplate area, a spinal pressure loading value based on body weight of the vertebrate subject; and
generating by computer-implemented generation from the spinal pressure loading value, a prognostic characterization of the susceptibility of the vertebrate subject to structural spine pain.

2. The method of claim 1, comprising generating the spinal pressure loading value for each of multiple spinal axial vertebra area loci.

3. The method of claim 2, wherein said loci are spinal lumbar region loci.

4. The method of claim 1, wherein the image comprises an axial image.

5. The method of claim 1, wherein the image comprises multiple images.

6. The method of claim 1, wherein the image is generated by an imaging apparatus comprising an image scanner generating said data of the image in digitized form.

7. The method of claim 1, wherein the proxy endplate area is determined by calculation of a product of disc area of a disc at the spinal axial vertebra area locus and a ratio of vertebral endplate diameter to disc diameter at the spinal locus.

8. The method of claim 1, wherein the spinal pressure loading value is determined by calculation of the ratio of body weight of the vertebrate subject to the proxy endplate area.

9. The method of claim 1, wherein the proxy endplate area is determined by calculation of a product of disc area of a disc at the spinal axial vertebra area locus and a ratio of vertebral endplate diameter to disc diameter at the spinal axial vertebra area locus, and wherein the spinal pressure loading value is determined by calculation of the ratio of body weight of the vertebrate subject to the proxy endplate area, optionally as age-adjusted for the vertebrate subject.

10. The method of claim 1, further comprising at least one of prescribing and conducting of therapeutic intervention comprising treatment of the vertebrate subject, based on the prognostic characterization of the susceptibility of the vertebrate subject to structural spine pain.

11. The method of claim 1, wherein the vertebrate subject comprises a human subject.

12. The method of claim 1, wherein generating by computer-implemented generation from the spinal pressure loading value, a prognostic characterization of the susceptibility of the vertebrate subject to structural spine pain, comprises generating said prognostic characterization in a report comprising at least one of: (i) the vertebrate subject's history of structural spine pain, (ii) correlations of variance of prognosed structural spine pain with future weight of the vertebrate subject, (iii) dietary recommendations, and (iv) lifestyle recommendations.

13. A non-transitory computer-readable storage medium storing instructions executable by a computer system to prognostically characterize susceptibility of the vertebrate subject to structural spine pain, the non-transitory computer-readable storage medium storing instructions to conduct the method according to claim 1.

14. An imaging and computational system for determining susceptibility of a vertebrate subject to structural spine pain, said system comprising:
an imaging apparatus configured to generate an image of a spinal axial vertebra area locus of the vertebrate subject; and a computer or other processor programmably configured to determine from data of the image generated by the imaging apparatus, a proxy endplate area of the spinal axial vertebra area locus, to determine from the proxy endplate area a spinal pressure loading value based on body weight of the vertebrate subject, and to responsively generate from the spinal pressure loading value, a prognostic characterization of the susceptibility of the vertebrate subject to structural spine pain.

15. The system of claim 14, further comprising a database arranged to receive imaging data from the imaging apparatus and computational data from the computer or other processor, for storage in the database, with the computer or other processor being configured to access the database and responsively generate the prognostic characterization of the susceptibility of the vertebrate subject to structural spine pain.

16. The system of claim 15, wherein the database contains historical spinal pressure loading values of the vertebrate subject.

17. The system of claim 15, wherein the database contains spinal pressure loading values of a vertebrate subject population, or a selected cohort or sub-population thereof.

18. The system of claim 17, wherein the database contains data concerning frequency and severity of structural spine pain events of the vertebrate subject population, or a selected cohort or sub-population thereof.

19. The system of claim 14, wherein the imaging apparatus is configured to generate axial images of the spinal axial vertebra area locus of the vertebrate subject.

20. The system of claim 14, wherein the computer or other processor is operatively linked in communication with a digital communications network.

21. A method of quantitatively determining susceptibility to structural spine pain of a human patient, comprising operating a digital computational apparatus in accordance with a programmed set of instructions to (i) determine from imaging data of an imaged spinal axial vertebra area locus and weight of the human patient, a spinal pressure at the axial vertebra area locus, and (ii) generate from the determined spinal pressure at the axial vertebra area locus a prognostic characterization of susceptibility of the human patient to structural spine pain.
